(19) United States
(12) Patent Application Publication
(10) Pub. No.: US 2026/0183560 A1
(43) Pub. Date: Jul. 2, 2026

(54) BODY IRRADIATION DEVICE FOR USE OF ACTINIC RADIATION ON A LIVING ORGANISM

(71) Applicant: KBL GmbH, Dernbach (DE)

(72) Inventor: Ulrich OBEL, Dachsenhausen (DE)

(21) Appl. No.: 19/129,765

(22) PCT Filed: Nov. 14, 2023
(86) PCT No.: PCT/EP2023/081795

§ 371 Date: May 14, 2025

(30) Foreign Application Priority Data
Nov. 15, 2022 (DE) 10 2022 212 158.9

(51) Int. Cl. A61N 5/06; A61N 5/00

(52) U.S. Cl. CPC A61N 5/0614; A61N 5/0616; A61N 2005/007; A61N 2005/0615; A61N 2005/0627; A61N 2005/0629; A61N 2005/0633; A61N 2005/0638; A61N 2005/0652; A61N 2005/0659; A61N 2005/0661; A61N 2005/0663

(57) ABSTRACT

The invention relates to a body irradiation device for an application of actinic radiation to a living organism, in particular a human being, which body irradiation device comprises at least one irradiation module, wherein the at least one irradiation module comprises: a circuit board, first LED sources of radiation, which are constructed so as to emit UV-A radiation, second LED sources of radiation, which are constructed so as to emit UV-B radiation, wherein the first and the second LED sources of radiation are arranged on the circuit board, wherein the circuit board comprises at least one first electric circuit and at least one second electric circuit, wherein the at least one first electric circuit connects the first LED sources of radiation among one another, wherein the at least one second electric circuit connects the second LED sources of radiation among one another, and wherein the circuit board has separate electrical connections for the at least one first electric circuit and the at least one second electric circuit

The invention relates to a body irradiation device for the application of actinic radiation to a living organism, in particular a human being, which body irradiation device comprises at least one irradiation module comprising a circuit board, LED sources of radiation, various electric circuits as well as their electrical connections.

Body irradiation devices for the human body are known, which are constructed, for example, in the form of a solarium with a surface on which to lie, a stand-up vertical tanning bed or a bed for treatment with red light. In such body irradiation devices, the body or parts of the body are exposed to a radiation spectrum in certain wavelength ranges in order to influence cosmetic aspects of the body, the well-being, the health or the regeneration of the body or of the person.

In this context, such body irradiation devices use, in general, low-radiation tubes, high-pressure radiation tubes or also high-pressure radiation lamps. In recent years, LED sources of radiation have also been increasingly used in body irradiation devices.

The document DE 20 2021 100 716 U1 relates to a body irradiation device for irradiating a body of a person or a part of a body of a person with radiation which is useful from a cosmetic-hygienic point of view. The body irradiation device comprises a source of irradiation with a base and at least one first LED chip, which can emit a first radiation spectrum with a first radiation peak, and at least one second LED chip, which can emit a second radiation spectrum with a radiation peak which is different from the first radiation peak, wherein the first LED chip and the second LED chip are arranged under a common lens in an LED housing and can be controlled separately.

DE 20 2021 104 364 U1 relates to a body irradiation device for an application of directed actinic radiation to a living organism, which body irradiation device comprises at least one irradiation module, and wherein the at least one irradiation module comprises: at least two LED sources of radiation, which generate the actinic radiation and which are arranged on a common support;
a panel that extends over the at least two LED sources of radiation;
spacers between the panel and the support, which spacers keep the panel and the support at a defined distance;
at least two piano-convex optical lenses which are firmly bonded to the panel in such a way that planar surfaces of the lenses face towards the support, wherein, in each case, one lens is set up and arranged in such a way that it at least substantially collimates or directs radiation emitted by an LED source of radiation.

It is an object of the invention to provide an improved body irradiation device for an application of actinic radiation to a living organism. In particular, it is an object of the invention to provide targeted irradiation with different types of actinic radiation, in particular UV-A radiation and UV-B radiation and preferably infrared radiation, by means of such a body irradiation device.

This object is achieved by the teaching of the independent claims. Advantageous embodiments are claimed in the dependent claims.

A second aspect of the invention relates to a body irradiation device for applying actinic radiation to a living organism, in particular a human being, wherein the body irradiation device comprises:
at least one irradiation module, wherein the at least one irradiation module comprises first LED sources of radiation which are constructed so as to emit UV-A radiation, and second LED sources of radiation which are constructed so as to emit UV-B radiation; and
control means for controlling the first LED sources of radiation and the second LED sources of radiation, in particular via their respective electric circuits, in such a way that a specific radiation intensity and/or a specific radiation dose of UV-A radiation and a specific radiation intensity and/or a specific radiation dose of UV-B radiation are emitted.

A second aspect of the invention relates to a body irradiation device for an application of actinic radiation to a living organism, in particular a human being, which body irradiation device comprises at least one irradiation module, wherein the at least one irradiation module comprises:
a circuit board,
first LED sources of radiation, which are constructed so as to emit UV-A radiation,
second LED sources of radiation, which are constructed so as to emit UV-B3 radiation,
wherein the first and the second LED sources of radiation are arranged on the circuit board, wherein the circuit board comprises at least one first electric circuit and at least one second electric circuit, wherein the at least one first electric circuit connects the first LED sources of radiation among one another, wherein the at least one second electric circuit connects the second LED sources of radiation among one another, and wherein the circuit board has separate electrical connections for the at least one first electric circuit and the at least one second electric circuit.

A third aspect of the invention relates to a method, in particular a non-therapeutic method, for the application of actinic radiation to a living organism, in particular a human being, by means of a body irradiation device, in particular according to any one of the preceding claims, wherein the method comprises the following process step:
controlling at least one first electric circuit and the at least one second electric circuit in such a way that a specific radiation intensity and/or a specific radiation dose of UV-A radiation and a specific radiation intensity and/or a specific radiation dose of UV-B radiation are emitted,
wherein the at least one first electric circuit connects first LED sources of radiation, which are constructed so as to emit UV-A radiation, among one another, and
wherein the at least one second electric circuit connects second LED sources of radiation, which are constructed so as to emit UV-B radiation, among one another.

In the sense of the invention, the term “actinic radiation” is intended to be understood to mean light or (broader) radiation of the entire electromagnetic spectrum (see definition in “Römpp Chemie-Lexikon”, Thieme Verlag, Stuttgart, Germany) which has a photochemical (including photobiochemical) effect and which, as the case may be, encompasses light/radiation of natural or artificial origin. In the claims and the description, the terms “actinic light” or “actinic radiation” are used for light or radiation of artificial origin, preferably light/radiation that is emitted by sources of radiation in a body irradiation device.

In a preferred embodiment of the body irradiation device, which may be implemented separately or together with one or two or more or all of the other features of the invention, the actinic radiation may be actinic radiation of a broad wavelength range. Alternatively, although also preferred, the actinic radiation may be actinic radiation of a narrow wavelength range or even actinic radiation of a very specific wavelength or a plurality of specific wavelengths. This is known to the skilled person, who, in accordance with the requirements of the individual case, can select the wavelength(s) or wavelength ranges or wavelength bands that are to be used.

In the sense of the invention, UV-B radiation is actinic radiation, preferably with a wavelength in the range from 280 to 315 nm.

In the sense of the invention, UV-A radiation is actinic radiation, preferably with a wavelength in the range from 315 to 400 nm.

In the sense of the invention, short-wave UV-B radiation is actinic radiation in the wavelength range from about 298 to 315 nm.

UV-B radiation promotes in particular the formation of new pigments, in particular the formation of melanin. UV-A radiation promotes in particular the tanning of the pigments, in particular the conversion of melanin. Short-wave UV-B radiation promotes in particular the vitamin D biosynthesis of the vitamin D precursors in human skin.

In the sense of the invention, IR radiation (infrared radiation) is actinic radiation, preferably in a wavelength band from 400 nm, in particular greater than 550 nm to 850 nm. In particular, IR radiation promotes biosynthesis of useful compounds for the care, the rejuvenation and the regeneration of the skin, such as for example collagen, elastin, keratin and hyaluronic acid.

In the sense of the invention, the term “about” in the context of wavelength specifications means +/−2 nm. Such a wavelength band around the characteristic wavelength is preferably exhibited by monochromatic LEDs.

In the sense of the invention, an LED source of radiation preferably comprises a single LED chip or a plurality of LED chips. Alternatively or additionally, the LED source of radiation has a receptacle and/or an interconnection of the LED chip or LED chips. Here, LED stands for “light emitting diode”.

In the sense of the invention, a means is preferably constructed in terms of hardware and/or software, and may comprise in particular a processing unit, in particular a microprocessor unit (CPU), in particular a digital processing unit, in particular a digital microprocessor unit (CPU), preferably connected to a memory system and/or a bus system in terms of data and/or signal communication, and/or may comprise one or more programs or program modules. Further preferably, the CPU is constructed so as to execute instructions which are implemented as a program which is stored in a memory system, to acquire input signals from a data bus, and/or to output output signals to a data bus. A memory system preferably comprises one or more storage media, in particular different storage media, in particular optical media, magnetic media, solid state media and/or other non-volatile media. The program may be of such nature that it embodies the methods described herein, or is capable of executing them, such that the CPU can execute the steps of such methods. In particular, in the sense of the invention, a control means is a control facility or a control device implemented in software.

In the sense of the invention, the term UV radiation encompasses UV-A radiation and UV-B radiation.

In the sense of the invention, the term radiation intensity is preferably synonymous with the term irradiance.

In the sense of the invention, the term “certain” preferably means predefined.

The invention is based on the realization that the use of UV-A LEDs and UV-B LEDs as sources of radiation makes a targeted emission in the UV spectrum and in the UV-B spectrum possible.

The invention makes it possible to arrange a large number of LED sources of radiation of the UV-A spectrum and of the UV-B spectrum in a comparatively small space in such a way that, on the one hand, a homogeneous field of radiation can be generated in relation to the different radiation spectra and, on the other hand, LED sources of radiation with different radiation spectra can be controlled separately. In this way, emission spectra can be combined in a controlled manner and can also be controlled with regard to the respective intensities emitted, and also with regard to the total radiation doses of the different radiation spectra of the LEDs that are emitted.

In this context, the radiation intensity and/or radiation dose emitted by the first sources of radiation and the radiation intensity and/or radiation dose emitted by the second sources of radiation can preferably be changed individually by the control means.

The invention makes it possible to use different temporal sequences of the photobiological effects and to separate these photobiological effects in time. In this way, for example, formation of pigment can be treated separately from tanning of pigment. Different scenarios of irradiation can also be put into effect by a user.

In addition, by providing a plurality of electric circuits of the same radiation spectrum, different areas of the body or of a body part can be controlled differently in dependence upon the respectively desired effect and the photobiological sensitivity of the user. In particular, an area of a user's face can be irradiated differently to the rest of the body.

In an advantageous embodiment, the at least one irradiation module further comprises:
at least one first circuit board on which the first LED sources of radiation are arranged; and
at least one second circuit board on which the second LED sources of radiation are arranged;
wherein the first circuit board has first regions and the second circuit board has second regions, wherein the first regions overlap with the second regions, and wherein a first LED source of radiation is arranged in at least one first region and a second LED source of radiation is arranged in at least one second region.

By providing different circuit boards for the different LED sources of radiation, these can be controlled separately from each other. In addition, the individual types of LED sources of radiation can be replaced separately from one another by changing individual circuit boards. This is of advantage in particular as the UV-B LEDs have a shorter service life than UV-A LEDs. In particular, the UV-B LEDs have a more significant decline in terms of power as a function of the time they have been in operation than is the case for the UV-A LEDs. Due to the overlapping areas of the circuit boards and a corresponding arrangement of sources of radiation in these areas, it can nevertheless be ensured that a sufficiently homogeneous field of UV-A radiation and a sufficiently homogeneous field of UV-B radiation are generated in the same spatial section.

In a further advantageous embodiment, the at least one first circuit board and the at least one second circuit board are arranged substantially in the longitudinal direction of the body irradiation device in an alternating manner. This also ensures that a sufficiently homogeneous field of UV-A radiation and a sufficiently homogeneous field of UV-B radiation are generated in the longitudinal direction of the body irradiation device.

In a further advantageous embodiment of the body irradiation device, the first circuit board has at least one first electric circuit and the second circuit board has at least one second electric circuit, wherein the at least one first electric circuit connects the first LED sources of radiation among one another, wherein the at least one second electric circuit connects the second LED sources of radiation among one another, and wherein the circuit boards have separate electrical connections for the at least one first electric circuit and the at least one second electric circuit.

In this way, an improved controllability of the different types of LED sources of radiation can be achieved.

In a further advantageous embodiment, the body irradiation device further comprises an exposure tunnel in which a user can lie down in order to be irradiated with actinic radiation, wherein the exposure tunnel is closed by substantially pivoting an upper part of the body irradiation device towards a lower part of the body irradiation device,
wherein the lower part of the body irradiation device has an at least substantially transparent surface, under which irradiation modules are arranged; and
wherein irradiation modules are also arranged on the upper part.

This construction is particularly advantageous when a user is to receive a full-body treatment while lying down.

In an advantageous embodiment, the body irradiation device has a greater number of first LED sources of radiation than second LED sources of radiation.

In order to achieve a lasting tanning effect, it is of advantage to generate a higher radiation intensity in the range of UV-A than in the range of UV-B. In particular, with the same nominal (physical) radiation intensity as UV-A, UV-B has a higher erythema effective radiation intensity on the skin of a user. For this reason, in order to achieve a photobiological effect, it is of advantage to provide more emitted UV-A radiation intensity than UV-B radiation intensity. Among other things, this can be achieved by the respective number of LED sources of radiation in the respective wavelength range.

In a further advantageous embodiment of the body irradiation device, the number of first LED sources of radiation is selected in such a way that an operating voltage of the at least one first electric circuit does not exceed about 70 V, preferably about 60 V, more preferably about 48 V and still more preferably about 36 V. Alternatively or additionally, the number of second LED sources of radiation is also selected in such a way that an operating voltage of the at least one second electric circuit does not exceed about 70 V, preferably about 60 V, more preferably about 48 V and still more preferably about 36 V.

This means that no or only insignificant separate insulation needs to be provided for the electric circuits. This simplifies the manufacture of the irradiation modules and makes them less expensive.

In a further advantageous embodiment of the body irradiation device, the first LED sources of radiation and the second LED sources of radiation are arranged offset. This also makes it possible to achieve a particularly homogeneous field of radiation of the irradiation modules.

In a further advantageous embodiment of the body irradiation device, the circuit board has separate electrical connections for each of the first electric circuits and/or separate connections for each of the second electric circuits.

As a result of this, each of the electric circuits can be controlled separately.

In a further advantageous embodiment of the body irradiation device, the first LED sources of radiation cover different bands, in particular frequency bands and/or wavelength bands, of the UV-A spectrum, wherein the first electric circuits connect first LED sources of radiation of each of a single defined band of the UV-A spectrum among one another and/or wherein the second LED sources of radiation cover different bands of the UV-B spectrum, wherein the second electric circuits connect second LED sources of radiation of each of a single defined band of the UV-B spectrum among one another.

As a result of this, any number of wavelength ranges of LEDs can be combined with one another in individual electric circuits. Depending on the activation or control of the individual electric circuits and the radiation spectrum that is associated therewith, different effects and/or types of therapy can be generated. As a result of this, for example, body irradiation devices, in particular solariums, of different UV classes can be realized in one device. Preferably, in this way, several devices can be realized in a single body irradiation device equipped with a fixed, that is non-variable, configuration of irradiation sources.

In a further advantageous embodiment of the body irradiation device, the second LED sources of radiation are configured to emit UV-B radiation from the following group of bands: about 297 nm, about 308 nm, about 311 nm, about 312 nm, and/or about 280 nm to about 315 nm.

All of these wavelengths produce a photobiological effect in human beings. In particular at a wavelength of 308 nm, a strong photobiological effect can be achieved with a low radiation intensity. Preferably, this is where a peak in the radiation intensity of the second LED sources of radiation is located.

In a further advantageous embodiment, the body irradiation device further comprises: third sources of radiation, which are set up to emit further actinic radiation, in particular IR radiation,
wherein the circuit board has at least one third electric circuit, wherein third electric circuits connect third sources of radiation among one another, and wherein the circuit board additionally has separate electrical connections for the at least one third electric circuit.

By providing means for emitting further types of actinic radiation, further photobiological effects can be activated by the body irradiation device.

Preferably, the first electric circuit exclusively connects first sources of radiation, the second electric circuit exclusively connects second sources of radiation, and/or the third electric circuit exclusively connects third sources of radiation.

In a further advantageous embodiment of the body irradiation device, at least two electric circuits cross each other on the circuit board, with one electric circuit having a bridge in each case. As a result of this, it is possible to achieve particularly homogeneous radiation distribution in relation to the LED sources of radiation with different radiation spectra. In particular, these can be arranged in an alternating manner in the direction of a radiation surface.

In a further advantageous embodiment of the body irradiation device, the irradiation module has a transparent panel which extends over the first LED sources of radiation and the second LED sources of radiation, wherein the panel is spaced apart from the circuit board, and at least one side of the panel, in particular the side of the panel which faces away from the circuit board, is sanitized.

In addition, the satin finish of the transparent panel also allows a diffusion of the light emitted by the LED sources of radiation to be achieved. This also contributes to a particularly homogeneous irradiation of the body. Preferably, the panel is therefore the only optical element of the at least one irradiation module. The elimination of further optical elements such as lenses or collimators as well as their installation reduces the manufacturing costs of the irradiation modules.

In a further advantageous embodiment of the body irradiation device, the panel is a glass panel.

Glass has a good durability with respect to UV radiation.

In a further advantageous embodiment of the body irradiation device, a radiation angle of the first LED sources of radiation and/or the second LED sources of radiation does not exceed about 50°, preferably about 40°, more preferably about 30° and is most preferably about 45°.

By using LED sources of radiation, the radiation angle of which is comparatively small, a particularly simple construction without reflector collimators and without lenses for collimating the emitted radiation can be realized, which nevertheless achieves a good homogeneity of the irradiation, that is, a homogeneous distribution of the radiation dose on a surface to be irradiated.

In a further advantageous embodiment of the body irradiation device, the irradiation module further has an at least partially transparent plastic panel which covers the panel, in particular on the side which faces towards the circuit board, and which has recesses in the region of the first LED source of radiation and/or in the region of the second LED source of radiation.

As a result of this, depending on the design of the plastic panel, certain areas of the body can be shaded.

Preferably, the plastic panel comprises a fluorescent material, and in particular it is coated with the fluorescent material. In this case, the plastic panel serves as an optical control function for the user, who will find it difficult to perceive the UV radiation, or, for example in the short-wave UV-B range, will not be able to perceive the UV radiation at all. The fluorescent plastic panel signals to the user whether radiation that is potentially harmful to the eyes or the skin is present. Here, the fluorescent material converts the UV radiations into visible light, at least in part.

In a further advantageous embodiment of the body irradiation device, the panel, in particular on the side which faces away from the circuit board, has an engraving in the region of the first LED source of radiation and/or in the region of the second LED source of radiation, preferably in the form of a ring, more preferably in the form of a plurality of concentric rings.

The engraving(s) result(s) in an element that is particularly suitable for the control function, which element lights up when visible light falls onto the engraving(s). This increases the safety of the user.

In a further advantageous embodiment of the body irradiation device, the first LED sources of radiation and the second LED sources of radiation are controlled in such a way that the radiation intensity of the first LED sources of radiation of the at least one first electric circuit and/or a radiation intensity of the second LED sources of radiation of the at least one second electric circuit varies over time.

By varying the irradiation over time, different temporal sequences of the photobiological effects can be used, and these photobiological effects can be separated in time. In this way, for example, irradiation with UV-B radiation can take place first, in order to promote the formation of pigment, and then irradiation with UV-A radiation can take place, in order to induce tanning of the pigment.

In a further advantageous embodiment, the body irradiation device has a sensor which is set up to measure at least one physiological parameter, in particular a pigmentation and/or a reaction of the skin of the living organism to an irradiation dose, wherein the first LED sources of radiation and the second LED sources of radiation are controlled in such a way that the radiation intensity or radiation intensities varies or vary as a function of the at least one physiological parameter.

By taking physiological parameters into account, a treatment process can be individually tailored to a user. In addition, it is possible for a user to set the desired result of a treatment, and the irradiation is adjusted accordingly. Such irradiation results can be, for example, a pre-tanning, a color or a degree of tanning.

In a further advantageous embodiment, the body irradiation device has a user interface which is set up in such a way that a radiation intensity of the UV-A radiation to be emitted and/or a radiation dose of the UV-A radiation to be emitted and a radiation intensity of the UV-B radiation to be emitted and/or a radiation dose of the UV-B radiation to be emitted can be set by means of the user interface, in particular individually, as a function of a maximum permissible erythema effective radiation intensity of UV radiation, and wherein the first LED sources of radiation and the second LED sources of radiation (5) are controlled on the basis of a selection, at the user interface, of the radiation intensity of the UV-A radiation to be emitted and/or the radiation dose of the UV-A radiation to be emitted and the radiation intensity of the UV-B radiation to be emitted and/or the radiation dose of the UV-B radiation to be emitted.

The use of UV LEDs as sources of radiation makes it possible to set the irradiation intensity individually. The user interface allows a user to specify the UV radiation intensity to which they are to be exposed. They can also set the radiation spectrum with which they are to be irradiated. In this context, the maximum permissible erythema effective radiation intensity serves as a reference value for setting the radiation intensity. This can be a value specified by law or a value that is freely selected within legal limits.

In a further advantageous embodiment of the body irradiation device, a temporal course of the radiation intensity of the UV-A radiation to be emitted and a temporal course of the radiation intensity of the UV-B radiation to be emitted can additionally be set via the user interface, wherein the first LED sources of radiation and the second LED sources of radiation are additionally controlled on the basis of a selection of a temporal course.

By taking into account a temporal course of an irradiation, different temporal sequences of the photobiological effects can be used, and these photobiological effects can be separated in time. In this way, for example, irradiation with UV-B radiation can take place first, in order to promote the formation of pigment, and then irradiation with UV-A radiation can take place, in order to induce tanning of the pigment.

In a further advantageous embodiment, the body irradiation device further has a user interface,
wherein at least one scenario of irradiation is stored in the means for controlling, for which a maximum permissible erythema effective radiation intensity of UV radiation is defined and which comprises a plurality of irradiation profiles,
wherein the plurality of irradiation profiles each define a radiation intensity of the UV-A radiation to be emitted and/or a radiation dose of the UV-A radiation to be emitted and a radiation intensity of the UV-B radiation to be emitted and/or the radiation dose of the UV-B radiation to be emitted as a function of the maximum erythema effective radiation intensity of UV radiation,
wherein the user interface is set up in such a way that the plurality of irradiation profiles can be selected, and
wherein the first LED sources of radiation and the second LED sources of radiation are controlled on the basis of a selection, at the user interface, of one of the plurality of radiation profiles.

Preferably, a plurality of scenarios of irradiation are stored in the means for controlling, which scenarios of irradiation can be selected by means of the user interface, wherein, for each scenario of irradiation, a different maximum permissible erythema effective radiation intensity of UV radiation is defined. Further preferably, the irradiation profiles additionally define, via the user interface, a temporal course of the radiation intensity of the UV-A radiation to be emitted and a temporal course of the radiation intensity of the UV-B radiation to be emitted.

The use of UV LEDs as sources of radiation makes it possible to define irradiation profiles, which define the radiation spectrum in the radiation intensity in individual regions of the irradiation spectrum. In addition, a temporal course of an irradiation can also be defined by irradiation profiles. This makes the operation much easier when a user is setting up the treatment and thus ensures maximum efficiency of the treatment and at the same time increases safety. If there are several scenarios of irradiation, these can be used to set the boundary conditions for the treatment with the various irradiation profiles. For example, the scenarios of irradiation can be used to set the maximum permissible erythema effective radiation intensity, which serves as a reference. In addition, the scenarios of irradiation and irradiation profiles can preferably be used to set the mood or atmosphere of a treatment with actinic radiation. In this context, the following parameters are possible: temperature in the treatment room, light color in the treatment room, background noise in the treatment room, scent in the treatment room, fog generation in the treatment room, ventilation in the treatment room and/or an admixture of warming infrared radiation to the UV radiation. The scenario of irradiation generally specifies which parameters are activated and which value ranges of the parameters are possible. The irradiation profiles then define specific values or temporal courses of values of the parameters.

The temporal courses of the irradiation profiles can extend over a single treatment session or several treatment sessions, in particular over several days. This can be advantageous in terms of a controlled formation of pigment, for example.

In a further advantageous embodiment of the body irradiation device, the user interface is constructed in such a way that the plurality of irradiation profiles can be selected, in particular continuously, between a maximum radiation profile with the highest radiation intensity to be emitted and/or with the highest radiation dose to be emitted and at least one radiation profile with a lower radiation intensity to be emitted and/or with a lower radiation dose to be emitted.

In this way, a particularly fine adjustment of the parameters of an irradiation profile can be carried out.

In a further advantageous embodiment of the body irradiation device, the radiation intensity of the UV-A radiation to be emitted and/or the radiation dose of the UV-A radiation to be emitted varies between different irradiation profiles differently than the radiation intensity of the UV-B radiation to be emitted and/or the radiation dose of the UV-B radiation to be emitted.

In this way, photobiological effects can be optimized.

In a further advantageous embodiment, the body irradiation device has at least two of the irradiation profiles from the following group of irradiation profiles: In The Morning—Intensive, In The Morning—Medium, In The Morning—Sensitive, At Midday—Intensive, At Midday—Medium, At Midday—Sensitive, In The Evening—Intensive, In The Evening—Medium and In The Evening—Sensitive, wherein, at least substantially, the maximum permissible erythema effective radiation intensity of UV radiation is reached at 100% UV-A radiation and 100% UV-B radiation, wherein the irradiation profiles are defined in the following table:

| | | In The | | In The |
|---|---|---|---|---|
| | | Morning | At Midday | Evening |
| Intensive | UV-A | about 75-85% | about 95-100% | about 75-85% |
| | UV-B | about 55-65% | about 95-100% | about 55-65% |
| Medium | UV-A | about 65-75% | about 85-95% | about 65-75% |
| | UV-B | about 45-55% | about 85-95% | about 45-55% |
| Sensitive | UV-A | about 55-65% | about 85-95% | about 55-65% |
| | UV-B | about 35-45% | about 75-85% | about 35-45% |

In a further advantageous embodiment of the body irradiation device, the at least one irradiation module further comprises:
third LED sources of radiation, which are constructed so as to emit red radiation and/or infrared radiation,
wherein the control means is arranged to control the third LED sources of radiation in such a way that a specific radiation intensity and/or a specific radiation dose of red radiation and/or infrared radiation is emitted.

By adding red and/or infrared radiation, the mood or atmosphere in the treatment room can be influenced in a targeted manner. In addition, infrared radiation can be used to achieve further photobiological effects.

In a further advantageous embodiment of the body irradiation device, the irradiation profiles from the group of irradiation profiles are further defined as follows, wherein, at least substantially, the maximum permissible radiation intensity of red radiation and/or infrared radiation is reached at 100% red radiation:

| | | In The | | In The |
|---|---|---|---|---|
| | | Morning | At Midday | Evening |
| Intensive | Red / | about 25-35% | about 95-100% | about 95-100% |
| | Infrared | | | |
| Medium | Red / | about 25-35% | about 95-100% | about 95-100% |
| | Infrared | | | |
| Sensitive | Red / | about 25-35% | about 95-100% | about 95-100% |
| | Infrared | | | |

In a further advantageous embodiment of the body irradiation device, the at least one irradiation module further comprises:
fourth LED sources of radiation, which are constructed so as to emit radiation in the visible spectrum and which are connected in series with the second LED sources of radiation in a second electric circuit.

Alternatively or additionally, the fourth LED sources of radiation have the same power supply as the second LED sources of radiation.

Alternatively or additionally, the fourth LED sources of radiation, together with the second LED sources of radiation, are controlled by the control means in such a way that the fourth LED sources of radiation are activated whenever the second LED sources of radiation are activated.

By one or all of these embodiments it can be ensured that the fourth LED sources of radiation are always activated together with the second LED sources of radiation, which emit UV-B radiation, and thereby emit light in the visible spectrum, which a user can perceive. Depending on the radiation spectrum, the UV-B radiation is barely visible or not visible at all to the user. Since the UV-B radiation can damage the skin and/or eyes of the user, the fact that the user can perceive when the second LED sources of radiation emit radiation is therefore an additional safety aspect. In addition, the fourth LED sources of radiation can also be used to create a defined mood or atmosphere in the treatment room. Preferably, the fourth sources of radiation therefore have a yellow color.

In a further advantageous embodiment of the body irradiation device, in a section of the irradiation module in the longitudinal direction of the body irradiation device in which a face of the living organism is arranged during use as intended, more first LED sources of radiation and/or second LED sources of radiation are arranged than in other sections of the body irradiation device.

This ensures that a user receives a higher radiation dose in the facial region than in the remaining regions of the body. At the same time, the LED sources of radiation in the facial region can be controlled substantially in the same way as in other regions of the body irradiation device, in particular with the same control current.

In a further advantageous embodiment of the body irradiation device, the number of second LED sources of radiation of the at least one irradiation module is selected in such a way that they can be operated at less than 70%, preferably less than 60%, most preferably at 50% of the rated current or the rated power of the second LED sources of radiation in order to emit the specific radiation intensity of UV-B radiation and/or to emit the specific radiation dose of UV-B radiation in a predetermined period of time.

UV-B LEDs are characterized by the fact that their power decreases significantly over their service life. By reducing the control current and/or the output power, the service life of the second LED sources of radiation can be increased. Ideally, a sufficient radiation intensity of the second LED sources of radiation is ensured over the entire service life of the body irradiation device.

In a further advantageous embodiment of the body irradiation device, the at least one irradiation module has a housing with end faces, wherein, on the end faces, regions which are permeable to air are provided for air supply, and, in a central area of the housing, an opening is provided for the discharge of air.

As a result of this, the first and second LED sources of radiation as well as other electronic and electrical components which are installed in the radiation module can be cooled efficiently without affecting the treatment room.

In a further advantageous embodiment of the body irradiation device, the opening of the housing of the at least one irradiation module is connected to an air duct in an upper part or in a lower part of the body irradiation device, and the air duct leads to a fan in the lower part of the body irradiation device.

As a result of this, it is not necessary to install one fan for each housing of the radiation module or several radiation modules. This reduces the energy consumption and the noise emission.

The features and advantages mentioned in relation to the first aspect of the invention also apply accordingly to the second and third aspects of the invention, and vice versa.

In an advantageous embodiment of the method, the electric circuits are controlled in such a way that a radiation intensity of the first LED sources of radiation of the at least one first electric circuit and/or a radiation intensity of the second LED sources of radiation of the at least one second electric circuit vary over time.

In this way, a separation of the photobiological effects in time can be utilized, for example with regard to the formation of pigment and the subsequent tanning of pigment.

In a further advantageous embodiment of the method, the radiation intensity or the radiation intensities vary in accordance with a predefined temporal profile.

In a further advantageous embodiment, the method comprises the following process step:
measuring at least one physiological parameter, in particular a pigmentation and/or a reaction of the skin to a radiation intensity and/or an irradiation dose of the living organism, wherein the radiation intensity or the radiation intensities and/or the irradiation dose varies/vary as a function of the at least one physiological parameter.

This makes it possible for a user to set the desired result of a treatment, and the irradiation is adjusted accordingly. Such irradiation results can be, for example, a pre-tanning, a color or a degree of tanning.

In a further advantageous embodiment of the method, the at least one first electric circuit and/or the at least one second electric circuit are controlled in such a way that the first LED sources of radiation emit about 98% and the second LED sources of radiation emit about 2% of a radiation intensity generated by the body irradiation device.

In this way, a particularly good tanning effect is achieved.

In a further advantageous embodiment of the method, the at least one first electric circuit and/or the at least one second electric circuit are controlled separately, in a pulsed manner.

In a further advantageous embodiment of the method, the at least one third electric circuit is also controlled in such a way that a specific radiation profile and/or a specific radiation dose is/are emitted.

Further preferably, the radiation intensity of the further actinic radiation from the third sources of radiation is also varied over time.

In a further advantageous embodiment, the method comprises the following process step: detecting a selection, in particular an individual selection, of a radiation intensity of the UV-A radiation to be emitted and/or a radiation dose of the UV-A radiation to be emitted and a radiation intensity of the UV-B radiation to be emitted and/or a radiation dose of the UV-B radiation to be emitted, as a function of a maximum permissible erythema effective radiation intensity of UV radiation;
wherein the first LED sources of radiation and the second LED sources of radiation are controlled on the basis of the selection of the radiation intensity of the UV-A radiation to be emitted and/or the radiation dose of the UV-A radiation to be emitted and the radiation intensity of the UV-B radiation to be emitted and/or the radiation dose of the UV-B radiation to be emitted.

In a further advantageous embodiment, the method comprises the following process step:
providing at least one scenario of irradiation for which a maximum permissible erythema effective radiation intensity of UV radiation is defined and which comprises a plurality of irradiation profiles;
detecting a selection of an irradiation profile from the plurality of irradiation profiles, each of which define a radiation intensity of the UV-A radiation to be emitted and/or a radiation dose of the UV-A radiation to be emitted and a radiation intensity of the UV-B radiation to be emitted and/or the radiation dose of the UV-B radiation to be emitted, as a function of the maximum permissible erythema effective radiation intensity of UV radiation;
wherein the first LED sources of radiation and the second LED sources of radiation are controlled on the basis of the selection of the one of the plurality of irradiation profiles.

In a further advantageous embodiment, the method comprises the following process step:
detecting a selection of a scenario of irradiation from a plurality of scenarios of irradiation, wherein, for each scenario of irradiation, a different maximum permissible erythema effective radiation intensity of UV radiation is defined.

In a further advantageous embodiment of the method, the second LED sources of radiation are operated at less than 70%, preferably less than 60% and most preferably at about 50% of the rated current or the rated power of the second LED sources of radiation.

In a further advantageous embodiment of the method, the first LED sources of radiation and the second LED sources of radiation in different sections in the longitudinal direction of the body irradiation device, in particular on different circuit boards, are controlled in such a way that different radiation intensities and/or radiation doses are emitted in different sections.

Further features and advantages will become apparent from the following description with reference to the figures. The figures show, at least partially schematically:

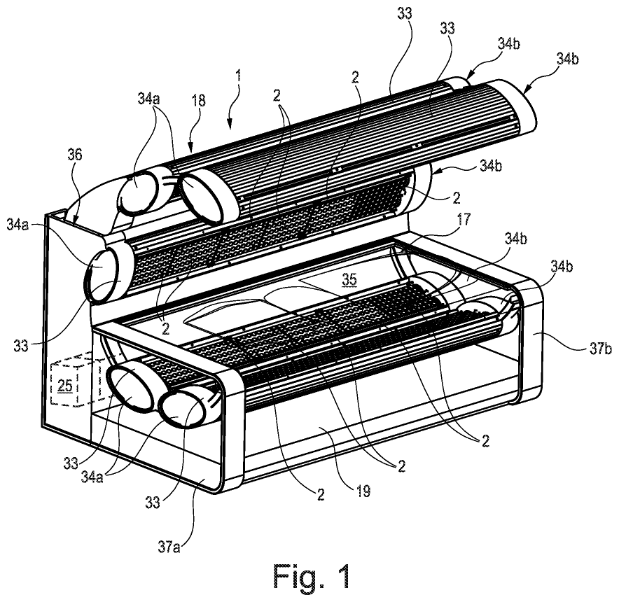

FIG. 1: shows a perspective view of a first example embodiment of a body irradiation device;

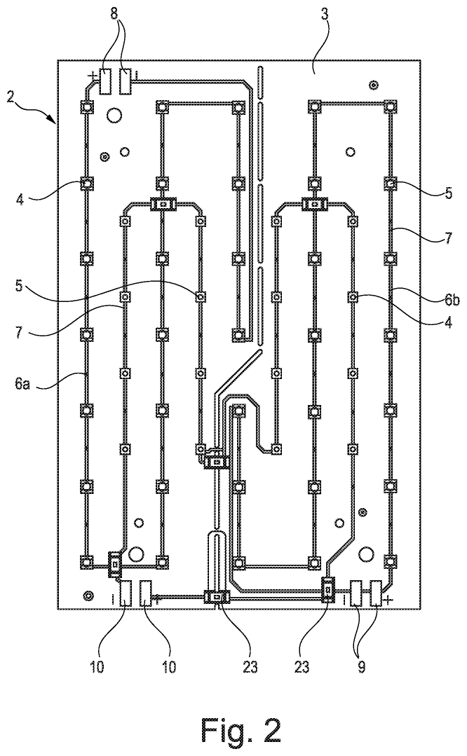

FIG. 2: shows a first example embodiment of an irradiation module;

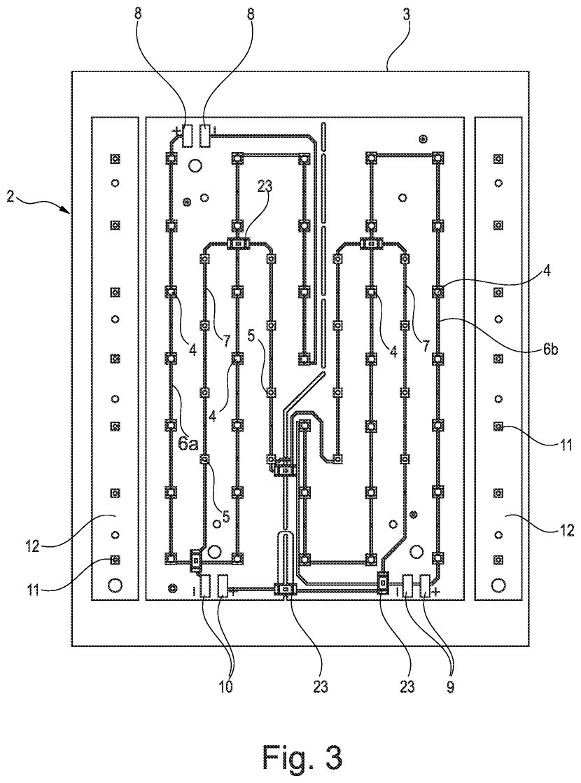

FIG. 3: shows a second example embodiment of an irradiation module;

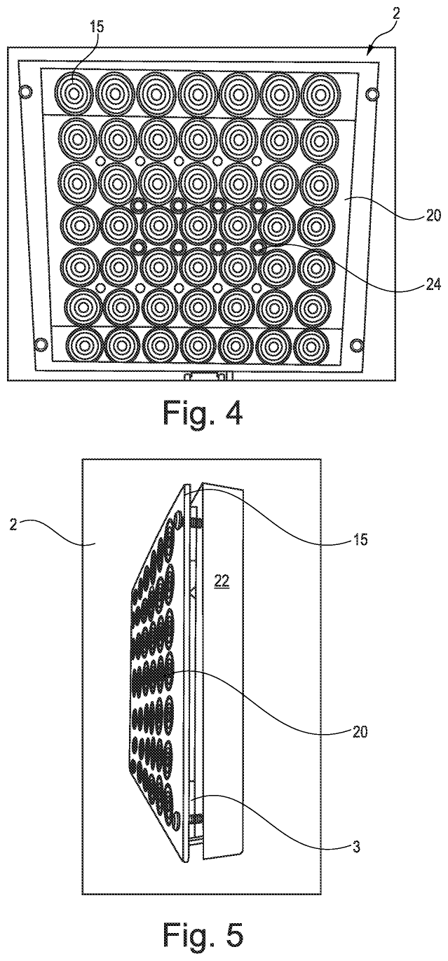

FIG. 4: shows a top view of a third example embodiment of an irradiation module;

FIG. 5: shows a lateral view of the third example embodiment of an irradiation module according to FIG. 4;

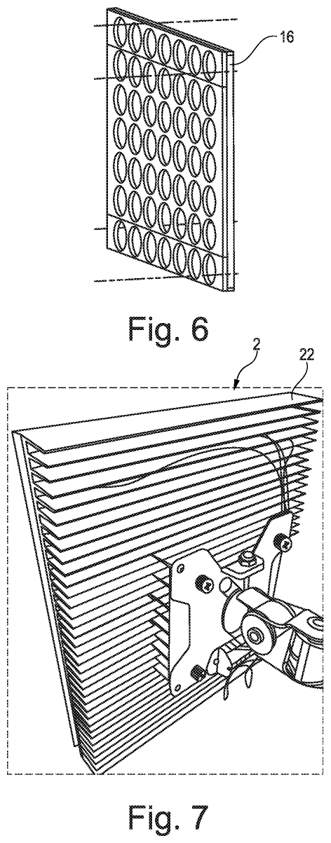

FIG. 6: shows a perspective view of a plastic panel of the third example embodiment according to FIGS. 4 and 5;

FIG. 7: shows a rear view of the third example embodiment of an irradiation module according to FIGS. 4 and 5;

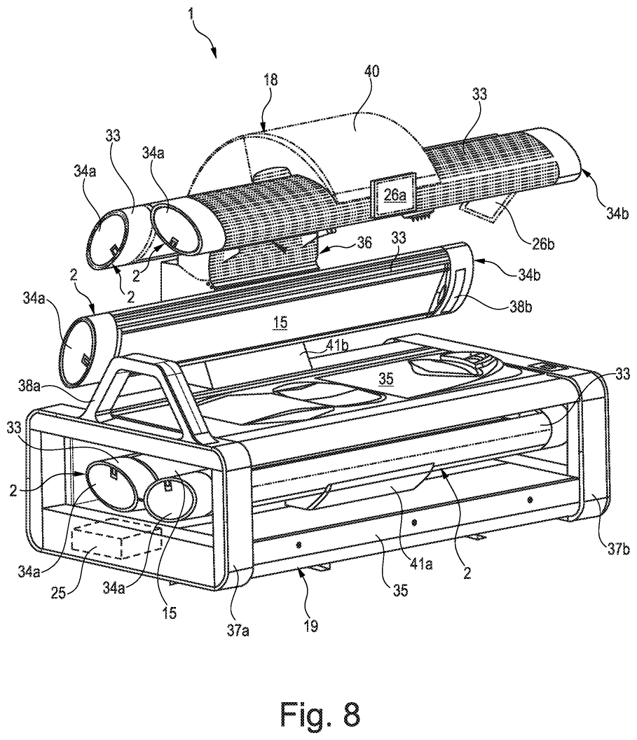

FIG. 8: shows a second example embodiment of a body irradiation device;

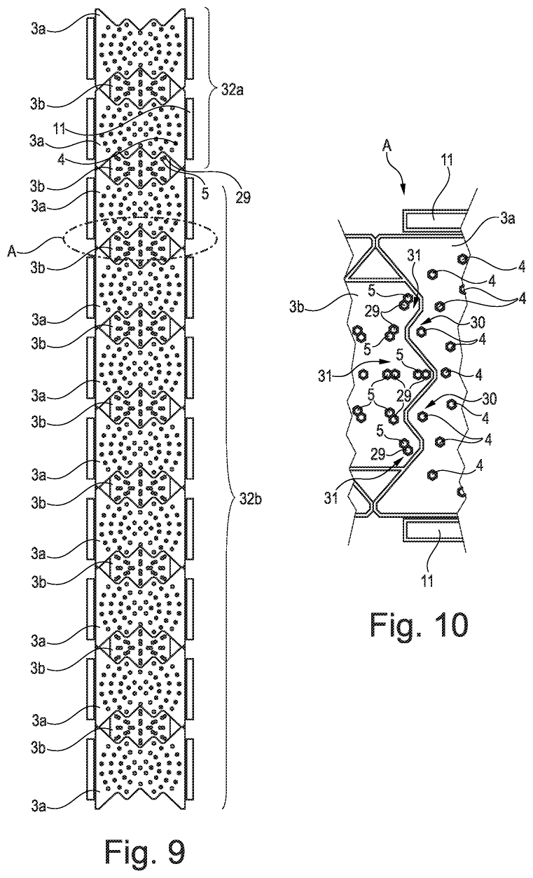

FIG. 9: shows a top view of the circuit boards of a fourth example embodiment of an irradiation module;

FIG. 10: shows an enlarged portion of the top view of the circuit boards according to FIG. 9;

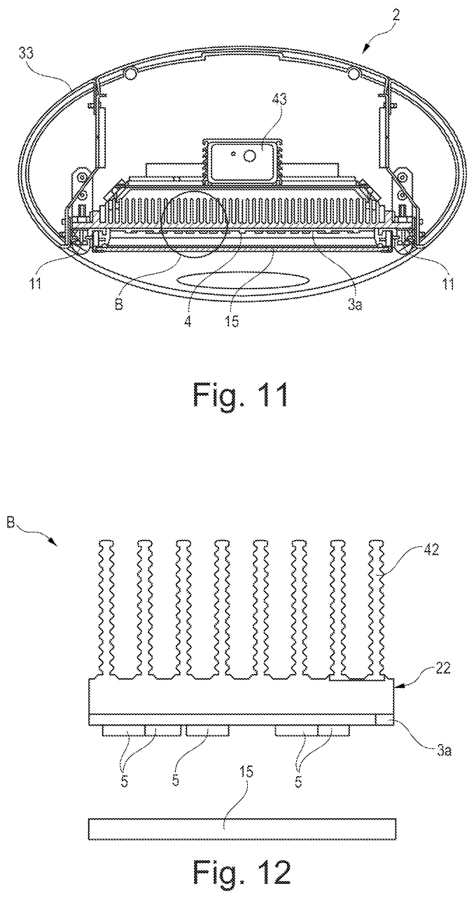

FIG. 11: shows a cross-sectional view of the fourth example embodiment of an irradiation module;

FIG. 12: shows an enlarged portion of the cross-sectional view of the fourth example embodiment of an irradiation module according to FIG. 11;

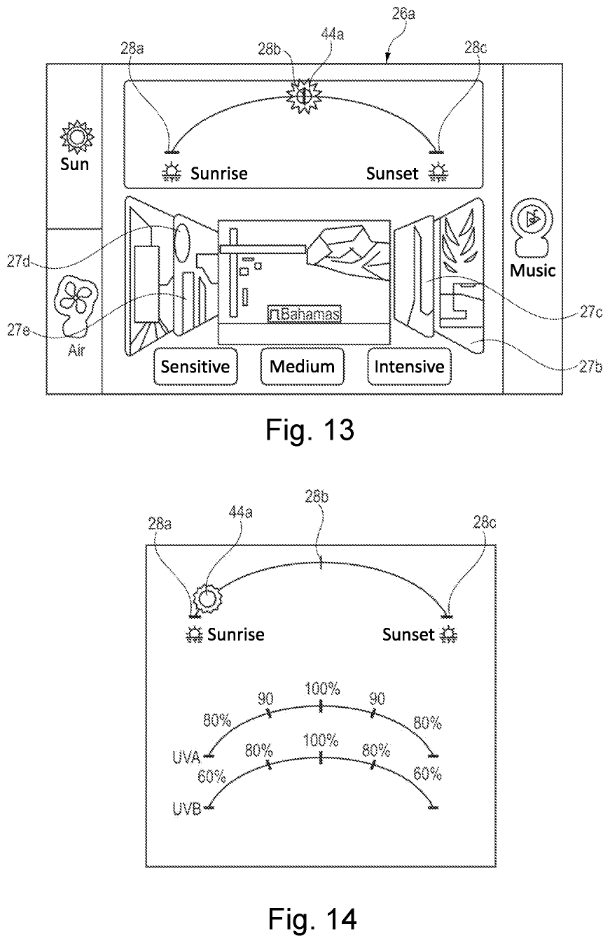

FIG. 13: shows a first view of a user interface;

FIG. 14: shows a representation of the principle of the function of the user interface according to FIG. 13;

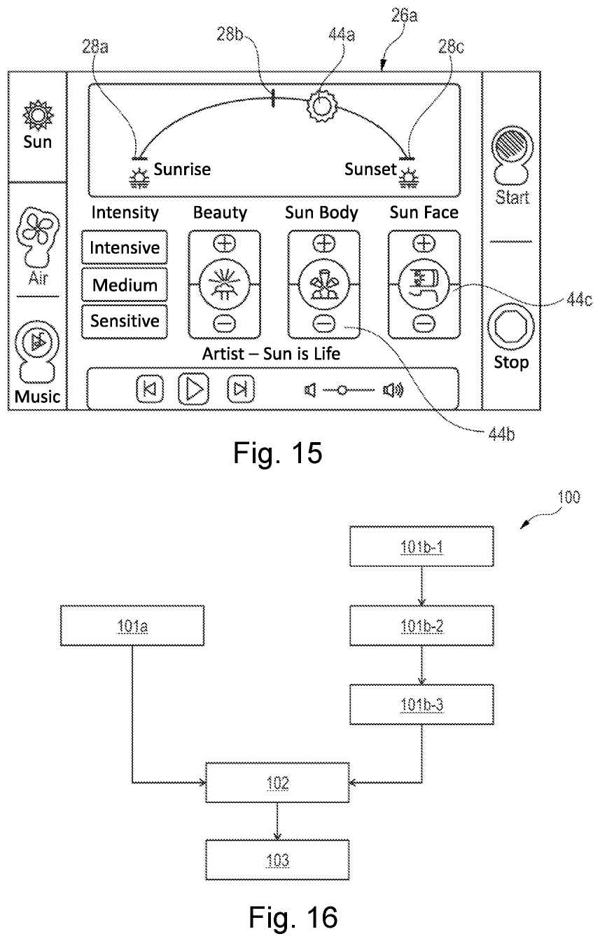

FIG. 15: shows a second view of the user interface according to FIG. 13; and

FIG. 16: shows a flow chart of an example embodiment of a method for applying actinic radiation to a living organism.

FIG. 1 shows an example embodiment of a body irradiation device 1.

This comprises an exposure tunnel 17 in which a user can lie down in order to be irradiated with actinic radiation.

Preferably, the exposure tunnel 17 is closed by pivoting an upper part 18 of the body irradiation device 1 substantially towards a lower part 19 of the body irradiation device 1 after the user has entered the exposure tunnel 17.

The lower part 19 of the body irradiation device 1 has an at least substantially transparent surface 35, under which irradiation modules 2 are arranged in two housings 33. Irradiation modules 2 are also arranged on the upper part 18.

In the example embodiment shown in FIG. 1, the lower part 19 has, in addition, a housing 33 with further irradiation modules 2, which are arranged above the transparent surface 35. In this case, the actual pivotable upper part 18 only comprises two housings 33 with irradiation modules 2, since a joint 36 for pivoting the upper part 18 is arranged between the two front housings 33 in FIG. 1 and the rear housing 33 above the transparent surface 35. The transparent surface 35, which is preferably formed by a glass or acrylic panel, is supported by two frame parts 37a, 37b.

Here, in the longitudinal direction of the exposure tunnel 17, a plurality of irradiation modules 2 are preferably arranged in a housing 33 of the irradiation modules 2, each of which can preferably be controlled separately. In this way, different regions of the body of the user, for example the head, torso, shoulders, legs, front and back, can be irradiated with different radiation spectra and/or radiation intensities or temporal radiation profiles.

Alternatively, it is also possible that a single radiation module 2 is arranged in a housing 33. In this case, individual LED sources of radiation 4, 5 or electric circuits 6A, 6B, 7, each with a plurality of LED sources of radiation 4, 5 are controlled separately.

Preferably, the body irradiation device 1 has a control means 25, which is used at least to control the LED sources of radiation 4, 5, 11, 29 (not shown). This control means 25 is preferably constructed as a software-implemented control facility, which is implemented in a computing unit of the body irradiation device 1, or is implemented as a stand-alone control unit.

In the end faces 34a, 34b, the radiation modules 2 preferably have regions which are permeable to air. Air for cooling the LED sources of radiation in the radiation modules 2 and the electronics required for operating the LED sources of radiation 4, 5, 11, 29 (not shown) can be drawn in through these end faces 34a, 34b. This air is blown out again via openings in the central region of the housing 33.

FIG. 2 shows a first example embodiment of an irradiation module 2.

Here, first LED sources of radiation 4 and second LED sources of radiation 5 are arranged on a circuit board 3. Here, the first LED sources of radiation 4 are electrically connected in series by two electric circuits 6A, 6B. In this context, the electric circuit 6A connects the LED sources of radiation 4 of that part of the circuit board 3 which, in FIG. 2, is on the left-hand side, while the electric circuit 6B connects the first UV-A sources of radiation of that part of the circuit board 3 which, in FIG. 2, is on the left-hand side. Both electric circuits 6A, 6B can be contacted separately via their respective contacts 8, 9 and can therefore also be controlled separately.

As a result of the UV-A sources of radiation being divided into two electric circuits 6a, 6b, the total operating voltage to be applied can be divided by two. In this way, if UV-A LED chips 5 with an operating voltage of 3.7 V are used, the total operating voltage for operating the electric circuit 6a, which connects 18 of the UV-A LED sources of radiation, can be limited to 66.6 V.

In addition, the irradiation module 2 has a further electric circuit 7, which electrically connects the second LED sources of radiation, which emit UV-B radiation, among one another in a series connection. This further electric circuit 7 crosses both the electric circuit 6A and the electric circuit 6B on the circuit board 3. Bridges 23 are arranged at each of the crossing points in order to lead the further electric circuit 7 across the electric circuits 6A, 6B.

In addition, the circuit board 3 has a predetermined breaking location in the central region of FIG. 2. This predetermined breaking location is also bridged by the further electric circuit 7 with the aid of bridges 23.

As can be seen in FIG. 2, the first LED sources of radiation 4, which emit UV-A radiation, are arranged in rows and columns. The second LED sources of radiation 5, which emit UV-B radiation, are respectively arranged offset to the first LED sources of radiation 4 and are also arranged in rows and columns.

The further electric circuit 7 can also be contacted separately and thus controlled via a further contact 10.

As a result of the UV-B LEDs 5 being arranged in spaces between the UV-A LEDs 4, a particularly homogeneous irradiation with both types of radiation can be ensured. On the one hand, a uniform irradiation intensity on an irradiation surface ensured, for example in the exposure tunnel 17, and, on the other hand, a comparatively large area can be irradiated with the irradiation module 2.

In case it is of advantage in a particular application, a single, correspondingly large irradiation module 2 can preferably also be used in order to irradiate an exposure tunnel 17 over its entire length.

FIG. 3 shows a second example embodiment of an irradiation module 2. This is substantially identical to the example embodiment of FIG. 2.

In contrast to the first example embodiment according to FIG. 2, however, the circuit board 3 has third LED sources of radiation 11 that emit red light or infrared light. These are also electrically connected to one another in a series connection by means of a separate electric circuit 12.

Here, two electric circuits 12 are preferably provided in the left-hand part and in the right-hand part according to FIG. 3. These electric circuits also preferably have separate contacts (not shown).

FIG. 4 shows a third example embodiment of an irradiation module 2. In this third example embodiment, a glass panel 15 extends over the circuit board on the side on which the LED chips or sources of irradiation 4, 5, 11 are arranged, which glass panel 15 is sanitized on the side which faces away from the circuit board 3. Further, rings 20 are engraved into the glass panel 15.

In this context, each of the concentric arrangements of rings 20 preferably covers one of the UV-A LED chips 4. Further concentric rings 24 preferably cover the UV-B LED chips 5.

In contrast to the first and the second example embodiments, only eight UV-B LED chips 5 are present in the third example embodiment. As a result of this, if UV-B LED chips 5 with an operating voltage of 5.5 V are used, the total operating voltage for operating the electric circuit 7, which connects the UV-B LED sources of radiation, can be limited to 44 V. Of course, the number of UV-B LED sources of radiation can be reduced accordingly in the first and the second example embodiments.

FIG. 5 shows a side view of the third example embodiment according to FIG. 4.

As can be seen in FIG. 5, the glass panel 15 is held at a distance from the circuit board 3 by fastening means (in FIG. 5, by screws). The element 22 in FIG. 5 is a heat sink. A further plastic panel 16 is preferably arranged between the glass panel 15 and the circuit board 3, which plastic panel 16 is further preferably adjacent the glass panel 15. This plastic panel 16 is preferably constructed so as to be fluorescent and, in an area that covers each of the LED chips or LED sources of radiation 4, 5, 11, preferably has circular recesses through which the radiation which is emitted by the LED chips 4, 5, 11 can freely hit the glass panel 15.

Such a plastic panel 16 is shown in FIG. 6.

FIG. 7 shows a rear view of the irradiation module 2. Here, cooling fins 42 of the heat sink 22 are visible.

FIG. 8 shows a second example embodiment of a body irradiation device 1.

This example embodiment of the body irradiation device 1 also comprises an upper part 18 and a lower part 19, which can be pivoted with respect to the lower part 19 in the region of the joint 36. Two irradiation modules 2 are attached to a pivot arm 40 of the upper part 18. Further, the upper part 18 has a first display 26a on the front side of the body irradiation device 1 and a second display 26b in the region of an end face of the body irradiation device 1.

The lower part 19 of the body irradiation device 1 also has irradiation modules 2. One of these irradiation modules 2 is arranged in a lateral region of the body irradiation device 1 above a transparent surface 35 of the lower part 19, on which a user lies, in the longitudinal direction of the body irradiation device 1, when the body irradiation device 1 is used as intended. Two further irradiation modules 2 are arranged below the transparent surface. The irradiation modules 2 are each held on support arms 41a, 41b. In addition, the support arm 41b carries the upper part 18 of the body irradiation device 1 via the joint 36. Further, the lower part 19 has a base 39, which is connected to the support arm 41a, 41b. The transparent surface 35, which is preferably formed by a glass or acrylic panel, is supported relative to the base 39 by two frame parts 37a, 37b.

A fan 38a for added comfort is attached to at least one of the frame parts 37a, 37b (in FIG. 8, this is frame part 37a), which fan 38a cools the user by means of an air jet when operated as intended. However, the fan 38a for added comfort can also be attached to a different element of the body irradiation device 1. Here, the fan 38a for added comfort is preferably formed as a kind of bracket, as is shown in FIG. 8, which bracket has a recess to the frame part 37a. Air is preferably blown out, substantially in the longitudinal direction of the body irradiation device 1, via slots in the bracket, so that a primary air flow is generated in the direction of the transparent surface or substantially parallel to it. This primary air flow preferably generates a secondary air flow, which flows through the recess. The primary air flow, together with the secondary air flow, cools the user so that a good cooling performance is achieved. In addition, the recess has the effect of creating an open feeling of space. As a result of this, a user can feel less cramped in the interior of the body irradiation device 1. Such a fan 38a for added comfort could also be attached to the body irradiation device 1 according to the first example embodiment according to FIG. 1 or to a different type of body irradiation device 1 in the sense of the present disclosure.

Preferably, the body irradiation device 1 has a control means 25, which is used at least to control the LED sources of radiation 4, 5, 11, 29. This control means 25 is preferably constructed as a software-implemented control facility, which is implemented in a computing unit of the body irradiation device 1, or as a stand-alone control unit. Preferably, this computing unit 25 or the control unit 25 is arranged in the base 39 of the lower part 19 of the body irradiation device 1, as is shown in FIG. 8.

In addition, at least one further fan 38b for added comfort is preferably provided in a head region of the body irradiation device 1 in the irradiation modules 2 which are arranged above the transparent surface 35.

The irradiation modules 2 have a housing 33, which is bounded, on a side which faces towards the transparent surface 35 by a transparent panel 15, from which the radiation emitted by the respective sources of radiation (not shown) can emerge.

Further, the radiation modules 2 preferably have regions in the end faces 34a, 34b which are permeable to air. Air can be drawn in through these end faces 34a, 34b for cooling the sources of radiation in the radiation modules 2 and the electronics which are required to operate the sources of radiation. Such air is extracted, via openings in the respective housings 33 through air ducts in the swivel arm 40 and in the support arms 41a, 41b, into the lower part of the body irradiation device 1, in particular into its base 39, by means of a fan (not shown) installed there.

By means of the displays 26a, 26b, which are constructed as user interfaces, a user can carry out the settings for the operation of the body irradiation device 1. Preferably, these displays are therefore constructed as touch-sensitive screens.

FIG. 9 shows a top view of the circuit boards 3a, 3b of a fourth example embodiment of the irradiation module 2.

These circuit boards 3a, 3b equipped with LED sources of radiation 4, 5, 29, in particular LED chips, are arranged in the interior of the housing 33 of the radiation modules 2 and, as will be explained below, are preferably covered by a panel 15, in particular a glass or acrylic panel, which, during use as intended, serves as a support surface for a user. In addition, further sources of radiation 11, in particular third LED sources of radiation, which are preferably constructed in the form of LED light strips, are arranged at the edge of the area that is bounded by the larger circuit boards 3a and which extends in the longitudinal direction.

As can be seen in FIG. 9, the first circuit boards 3a as well as the second circuit boards 3b preferably have a kind of butterfly shape, wherein, further preferably, two halves of each of the circuit boards 3a and 3b are axially symmetrical with respect to a central axis. As can be seen in FIG. 9, the contours of the first circuit boards 3a and of the second circuit boards 3b preferably complement each other in such a way that they can be arranged adjacent to each other in the longitudinal direction of an irradiation module 2.

First LED sources of radiation 5 are arranged on the first circuit boards 3a, which preferably have a larger surface area than the second circuit boards 3b. Here, the arrangement of the first LED sources of radiation 5 on the first circuit boards 3a preferably forms sectors of concentric rings. In this context, the second LED sources of radiation 5 and the fourth LED sources of radiation 29, which are arranged on the second circuit boards 3b, are preferably arranged in such a way that they substantially complement the sectors of the concentric rings which are formed by the first LED sources of radiation 4. In addition, further second LED sources of radiation 5 and fourth LED sources of radiation 29 are arranged along the axis of symmetry of the second circuit boards 3b. The second LED sources of radiation 5 and the fourth LED sources of radiation 29 are preferably arranged in pairs on the second circuit boards 3b, in particular adjacent to one another.

In the fourth example embodiment of the irradiation module 2 shown in FIG. 9, the irradiation module 2 comprises ten first circuit boards 3a and nine second circuit boards 3b.

Those two circuit boards 3a which are arranged in a section 32a, in which the face of a user is arranged when the body irradiation device 1 is used as intended, preferably have a greater number of first LED sources of radiation 4 than the first circuit boards 3a in the second section 32b. The second circuit board 3b, which is arranged in the first section 32a also has a greater number of second LED sources of radiation 5 than those second circuit boards 3b which are arranged in the second section 32b shown.

In the fourth example embodiment shown in FIG. 9, the first circuit boards 3a into the first section 32a each have 49 first LED sources of radiation 4, and the second circuit board 3b has 16 second LED sources of radiation 5 and the second circuit board 3b each has 16 fourth LED sources of radiation 29.

In the second section 32b, each of the first circuit boards 3a have 39 first LED sources of radiation 4, and each of the second circuit boards 3b has 12 second LED sources of radiation 5 and twelve fourth LED sources of radiation 29.

Preferably, the first LED sources of radiation 4 emit UV-A radiation and the second LED sources of radiation 5 emit UV-B radiation. This means that, preferably, the major portion of the radiation spectrum emitted by the first LED sources of radiation 4 is in the UV-A radiation range and the major portion of the radiation spectrum emitted by the second LED sources of radiation 5 is in the UV-B spectrum. The third LED sources of radiation 11 preferably emit red and/or infrared radiation. The fourth sources of radiation 29 preferably emit radiation in the visible spectrum, in particular in the yellow spectrum.

Preferably, the first LED sources of radiation 4 and the second LED sources of radiation 5 have a radiation angle of about 45°, that is, an angle of two times about 22.5° with respect to the surface normal.

FIG. 10 shows an enlarged view of an area A of FIG. 9.

As can be seen in FIG. 10, the first circuit board 3a and the second circuit board 3b preferably mesh with one another. In particular, the first circuit board 3a has first regions 30, which mesh with second regions 31 of the second circuit board 3b.

Here, first LED sources of radiation 4 are preferably arranged in the area of the first regions 30, and second LED sources of radiation 5 are preferably arranged in the area of the second regions 31, in particular as a pair with fourth LED sources of radiation 29. As a result of this overlapping of the first regions 30 and the second regions 31 and the first LED sources of radiation 4 and the second LED sources of radiation 5 respectively arranged in these regions 30 and 31, a comparatively homogeneous irradiation intensity of UV-A radiation and UV-B radiation can be achieved in the longitudinal direction of the irradiation modules 2, although only first LED sources of radiation 4, which emit UV-A radiation, and second LED sources of radiation 5, which emit UV-B radiation, are respectively arranged on the adjacent first circuit boards 3a and second circuit boards 3b arranged in this direction.

In addition, portions of two third LED sources of radiation 11 can be seen in FIG. 10, which, as has already been explained above, are preferably formed by LED light strips.

FIG. 11 shows a cross-sectional view of an irradiation module 2 according to the fourth example embodiment. Here, the cross-sectional view shows a cross-section at the level of a first circuit board 3a.

The circuit board 3a is preferably attached to the housing by means of fastening means (no reference sign). The same applies to the third LED sources of radiation 11, which are preferably supported by a further circuit board (no reference sign). The first LED sources of radiation 4 on the circuit board 3a are preferably covered by a panel 15, in particular a glass or acrylic panel, which is transparent. This panel 15 is also supported on the housing 33 of the radiation module 2 by means of fastening means (no reference sign).

A heat sink 22 is preferably arranged on the rear side of the circuit board 3a, which is opposite the panel 15. In turn, on the rear side of the heat sink 22, a power supply 43 for the first LED sources of radiation 4 of the first circuit board 3a is arranged on a further fastening element (no reference sign).

Due to the comparatively large air space in the irradiation module 2, all elements can be cooled well by means of air. As has already been described with reference to FIG. 8, such air is preferably drawn into the irradiation modules 2 via the end faces 34a, 34b (not shown) and then evacuated via an opening in the irradiation modules 2.

FIG. 12 shows an enlarged view of an area B of FIG. 11.

As can be seen in FIG. 12, the heat sink 22 preferably has cooling fins 42 in order to achieve a better cooling performance.

The panel 15 is preferably sanitized on one side, preferably on the side that is facing away from the first circuit board 3a.

Further, a distance between the panel 15 and the surface of the circuit board is between about 20 mm and about 30 mm, preferably between about 15 mm and about 10 mm, and most preferably about 13 mm. In this way, a particularly good scattering effect of the radiation emitted by the first LED sources of radiation and the second LED sources of radiation can be achieved, so that a particularly homogeneous distribution of radiation can be achieved on the surface of a user's body to be irradiated.

FIG. 13 shows a view of the first user interface 26a. Preferably, the second user interface 26b, which is shown in FIG. 8, can also be designed in an identical manner.

Preferably, the user interfaces 26a, 26b are constructed as touch-sensitive screens. However, also conceivable is any other type of user interface by means of which inputs by the user are possible.

As is shown in FIG. 13, the user interface 26a has a first slide controller 44, by means of which various irradiation profiles 28a, 28b, 28c can be set within a scenario of irradiation 27a, which is preferably also displayed further down on the user interface 26a. Further preferably, the respective scenario of irradiation 27a, 27b, 27c, 27d, 27e can also be selected by touching the respective graphic that represents the scenario of irradiation.

In addition, in the right-hand region of the user interface 26a, a setting mask of a stereo system preferably integrated in the irradiation device 1 can be selected, and in the left-hand region of the user interface 26a, a setting mask of the fan 38a, 38b for added comfort (not shown) as well as precisely the shown setting mask of the irradiation can be selected.

Each scenario of irradiation 27a, 27b, 27c, 27d, 27e preferably comprises at least two irradiation profiles 28a, 28b, 28c. These irradiation profiles 28a, 28b, 28c are preferably selected from the following group of irradiation profiles: In The Morning—Intensive, In The Morning—Medium, In The Morning—Sensitive; At Midday—Intensive, At Midday—Medium, At Midday—Sensitive; In The Evening—Intensive, In The Evening—Medium, In The Evening—Sensitive. Here, the irradiation profiles 28a, 28b, 28c mentioned above are defined in the following table, wherein, at least substantially, the maximum permissible erythema effective radiation intensity of UV radiation is reached at 100% UV-A radiation and 100% UV-B radiation. In this context, the maximum permissible erythema effective radiation intensity can be a value specified by law or can be defined individually, for example by an operator of the respective body irradiation device 1.

| | | In The | | In The |
|---|---|---|---|---|
| | | morning | At Midday | Evening |
| Intensive | UV-A | about 75-85% | about 95-100% | about 75-85% |
| | UV-B | about 55-65% | about 95-100% | about 55-65% |
| Medium | UV-A | about 65-75% | about 85-95% | about 65-75% |
| | UV-B | about 45-55% | about 85-95% | about 45-55% |
| Sensitive | UV-A | about 55-65% | about 85-95% | about 55-65% |
| | UV-B | about 35-45% | about 75-85% | about 35-45% |

In addition, the radiation profiles of the group mentioned above can be defined according to the following table, wherein visible radiation means in the visible range, in particular in the red spectrum, and l or in the infrared spectrum. Here, 100% radiation corresponds to a predefined value.

| | | In The | | In The |
|---|---|---|---|---|
| | | morning | At Midday | Evening |
| Intensive | Visible | about 25-35% | about 95-100% | about 95-100% |
| Medium | Visible | about 25-35% | about 95-100% | about 95-100% |
| Sensitive | Visible | about 25-35% | about 95-100% | about 95-100% |

Sensitive Visible about 25-35% about 95-100% about 95-100% In addition, by selecting a scenario of irradiation 27a, 27b, 27c, 27d, 27e, an off-set value can be defined, which changes the radiation intensity for the respective irradiation profiles 28a, 28b, 28c across the entire emission spectrum. This makes it possible, for example, to select irradiation profiles that are dependent on the time of day, as they are typical for different regions of the world.

Preferably, the irradiation profiles 28a, 28b, 28c are not only selectable in a discrete manner, but these can be continuously changed between a maximum value of the irradiation, which preferably represents the irradiation profile 28b, and minimum values of the irradiation intensities, as they are represented by the irradiation profiles 28a and 28c.

This method of controlling the irradiation intensity is shown in the representation of the principle of the function of the user interface 26a in FIG. 14. Here, the slide controller 44a can be moved along a curved line between the discrete scenario of irradiation In The Morning—Intensive on the left-hand side 28a via At Midday—Intensive at 28b to the discrete scenario In The Evening—Intensive 28c. Depending on the position of the slide controller 44a, the UV-A and UV-B irradiation then changes in accordance with the curve shown below in FIG. 14.

In this way, a continuous stepless adjustment of the scenario of irradiation 27a, 27b, 27c, 27d, 27e between the two discrete extreme scenarios In The Morning—Intensive and In The Evening—Intensive is possible.

In this context, preferably, the third LED sources of radiation 11 and/or the fourth LED sources of radiation 29 are used to generate a lighting mood that corresponds to the respective selected scenario of irradiation.

In addition, the radiation scenarios 27a, 27b, 27c, 27d, 27e and the radiation profiles 28a, 28b, 28c can preferably be used to set the mood or atmosphere of a treatment with actinic radiation. In this context, the following parameters are possible: temperature in the treatment room, light color in the treatment room, background noise in the treatment room, scent in the treatment room, fog generation in the treatment room, ventilation in the treatment room and/or an admixture of warming infrared radiation to the UV radiation.

The scenario of irradiation 27a, 27b, 27c, 27d, 27e preferably specifies, in general, which parameters are activated and which value ranges of the parameters are possible. The irradiation profiles 28a, 28b, 28c then preferably define specific values or temporal courses of values of the parameters.

FIG. 15 shows a second view of the first user interface 26a. As has already been explained with reference to FIG. 13, this view could also be displayed on the user interface 26b.

In contrast to the first view in FIG. 13, this view has a second slide controller 44b, with which the irradiation intensity can be adjusted in the section 32b of the irradiation modules 2, in which the body of a user is located during use as intended.

In addition, a third controller 44c can preferably be used to adjust the radiation intensity in the section 32a of the radiation modules 2 in which the face of a user is located during use as intended. In this way, the two sections 32a, 32b can be controlled independently of each other.

In the same manner as in the view shown in FIG. 13, selection options for selecting other masks of the control facility are shown in FIG. 15 in each of the left-hand and right-hand regions of the user interface 26a.

FIG. 16 shows an example embodiment of a method 100, in particular a non-therapeutic method, for the application of actinic radiation to a living organism. Preferably, a body irradiation device 1 as described with reference to the preceding figures and example embodiments is used here.

Preferably, a body irradiation device 1 as described with reference to the preceding figures and example embodiments is used here.

Here, the radiation intensity emitted by the first LED sources of radiation 4 and the radiation intensity emitted by the second LED sources of radiation 5 can be adjusted individually. In particular, the first LED sources of radiation 4 of the first circuit board 3a are connected to each other via a first electric circuit for this purpose, and the second LED sources of radiation 5 and the fourth LED sources of radiation 29 with the second circuit board are connected to each other via a further electric circuit. These electric circuits are preferably supplied separately by means of a separate power supply. Preferably, each individual circuit board 3a, 3b is supplied by means of a separate power supply. Alternatively, groups of first circuit boards 3a and groups of second circuit boards 3b or their respective electric circuits can also be supplied by means of a single power supply 43.

In a first process step 101a of the method 100, a selection of a radiation intensity of the UV-A radiation to be emitted and/or a radiation dose of the UV-A radiation to be emitted is preferably detected. At the same time, or independently thereof, a selection of a radiation intensity of the UV-B radiation to be emitted and/or a radiation dose of the UV-B radiation to be emitted is preferably detected. Preferably, the respective selection is detected via a user interface 26a, 26b, which is further advantageously constructed as a touch-sensitive screen. On such a touch-sensitive screen 26a, 26b, slide controllers 44a, 44b, 44c are provided, which allow the respective UV-A radiation to be emitted and the respective UV-B radiation to be emitted to be set jointly or individually.

In addition, a detection is preferably carried out as to whether different radiation intensities and/or radiation doses are to be emitted in different sections 32a, 32b of the body irradiation device 1. This can also be done via corresponding slide controllers 44a; 44b, 44c of a user interface 26a, 26b.

In this context, a user interface 26a, 26b is used to detect inputs by a user.

Preferably, the radiation intensity of UV-A radiation and UV-B radiation to be emitted and/or the radiation dose of UV-A radiation and UV-B radiation to be emitted is selected as a function of a maximum permissible erythema effective radiation intensity, and this maximum permissible erythema effective radiation intensity is stored in the means for controlling 25, in particular in the control device 25, via the computer-implemented control facility. The erythema effective radiation or power is preferably specified in terms of power per square meter [W/sqm] and takes into account the erythema effectiveness of the respective type of radiation. The term erythema effectiveness refers to the ability of ultraviolet radiation to cause sunburn in the skin after certain threshold values, such as for example the erythema threshold dose or the threshold exposure time have been exceeded. Due to the dependence of the sensitivity of the skin to erythema on the dose and wavelength, the erythema effectiveness of a source of UV radiation is determined by its spectral distribution and by its radiation intensity. For example, UV-B radiation has a higher erythema effectiveness than UV-A radiation. With regard to these photobiological effects, reference is also made to the standards IEC 60335-2-27 and DIN EN 60335-2-27.

In an alternative of the example embodiment, at least one scenario of irradiation 27a, 27b, 27c, 27d, 27e is made available for selection in a first partial step 101b-1, and is in particular made available for selection via a user interface 26a, 26b. These scenarios of irradiation 27a, 27b, 27c, 27d, 27e also define a maximum permissible erythema effective radiation intensity.

In addition, each scenario of irradiation 27a, 27b, 27, 27d, 27e preferably comprises a plurality of irradiation profiles 28a, 28b, 28c. The irradiation profiles 28a, 28b, 28c each define a radiation intensity of the UV-A radiation and of the UV-B radiation to be emitted and/or a radiation dose of the UV-A radiation and of the UV-B radiation to be emitted as a function of the maximum permissible erythema effective radiation intensity of UV radiation.

With different irradiation profiles 28a, 28b, 28c different intensities of the irradiation of a user are therefore also defined in the body irradiation device 1. In addition, the radiation doses of UV-A radiation and UV-B radiation to be emitted can also be determined via the radiation profiles 28a, 28b, 28c. Temporal courses of the UV-A radiation and the UV-B radiation can also be defined.

If third LED sources of radiation 11 and/or fourth LED sources of radiation 29 are present, then their radiation intensity to be emitted as well as temporal courses of the radiation intensity and a total radiation dose to be emitted can also be determined, by the radiation profiles 28a, 28b, 28c, with respect to these third sources of radiation 11 and/or these fourth LED sources of radiation 29, in particular with respect to red and/or infrared radiation and/or radiation in the visible spectrum.

In this way, a scenario of irradiation 27a, 27b, 27c, 27d, 27e in conjunction with the irradiation profiles 28a, 28b, 28c can be used to realize a wide variety of irradiation variants, which, in terms of the types of irradiation and moods are reminiscent of different geographical locations or reproduce these. Accordingly, it is possible, (as is shown in FIG. 13, for example) to store scenarios of irradiation such as the Bahamas, Paris, Berlin, the Cate d'Azur and the Canary Islands in the control means 25, each of which can be selected by a user. Preferably, the actinic radiation then emitted, as well as the mood or atmosphere, imitate these places.

In a second partial process step 101b-2, a selection, by a user, of the respective scenario of irradiation from the plurality of scenarios of irradiation 27a, 27b, 27c, 27d, 27e is detected.

This selection is preferably detected via the user interface 26a, 26b as well. On the basis of the selection of the scenario of irradiation 27a, 27b, 27c, 27d, 27e, the user is provided, preferably also via the user interface 26a, 26b, with a plurality of irradiation profiles 28a, 28b, 28c to choose from.

In a third partial process step 101b-3, a choice of the respective irradiation profile from the plurality of irradiation profiles 28a, 28b, 28c is then detected.

Accordingly, the body irradiation device 1 therefore preferably has the functions and corresponding means for the user to select such scenarios. In this context, it is conceivable, for example, that a specific geographical location on Earth is specified, as well as the time of day the irradiation of which is to be imitated, for example Malibu, June, midday or Mallorca, August, in the afternoon.

Preferably, the body irradiation device 1 has a location determination means for this purpose, for example a GPS module, in order to determine its location and to control the irradiation in accordance with this geographical location.

In a second process step 102, physiological parameters, in particular a pigmentation and/or a reaction of the skin to an irradiation dose, of the user are preferably measured. The radiation intensity respectively emitted by the LED sources of radiation 4, 5 can then be varied as a function of the at least one physiological parameter.

Alternatively or in addition, a radiation intensity actually emitted, in particular a radiation intensity of UV-A radiation and/or UV-B radiation, can also be measured in the second process step, and the radiation intensity can be varied as a function of the actual radiation intensities.

In a third process step 103, the LED sources of radiation are controlled in such a way that a specific radiation intensity, in particular in a time-dependent course, and/or a specific radiation dose are emitted within a predefined period of time. In particular, a UV-A radiation, a UV-B radiation and/or a red and/or infrared radiation respectively from the first LED sources of radiation 4, the second LED sources of radiation 5 and the third LED sources of radiation 11 can be emitted in a controlled manner in this way. In this context, the radiation intensity of the LED chips or LED sources of radiation 4 which emit UV-A radiation, and/or the radiation intensity of the LED chips or LED sources of radiation 5 which emit UV-B radiation, can preferably be varied over time.

Here, electric circuits 6a, 6b, 7, which connect the first LED sources of radiation 4, the second LED sources of radiation 5 and the third LED sources of radiation 11 with one another, are in particular controlled in such a way that the radiation intensity of the respective LED sources of radiation 4, 5, 11 varies over time.

Here, the specific temporal irradiation profiles 28a, 28b, 28c and/or the specific radiation dose can be set on the basis of the measurement of the at least one physiological parameter carried out in the second process step 102. In addition, the specific temporal irradiation profiles 28a, 28b, 28c and/or the specific irradiation dose can be set in advance on the basis of further criteria.

In addition, the second LED sources of radiation 5 are controlled in such a way that they are operated at less than 70%, preferably less than 60%, and most preferably at about 50% of the rated current or the rated power of the second LED sources of radiation 5.

In addition, the first LED sources of radiation 4 and the second LED sources of radiation 5 in different sections 32a, 32b in the longitudinal direction of the body irradiation device 1 are preferably controlled in such a way that different radiation intensities and/or radiation doses are emitted in the different sections 32a, 32b.

Since the body irradiation device 1 and the method 100 offer the possibility of varying different types of radiation, in particular UV-A radiation, UV-B radiation and red radiation or IR radiation, independently of one another over time, different scenarios of natural irradiation, in particular solar irradiation, can be imitated.

It is to be noted that the example embodiments are merely examples which are not intended to restrict the scope of protection, the application and the structure in any way. Rather, the preceding description will provide the person skilled in the art with a guideline for the implementation of at least one example embodiment, whereby various changes, in particular with regard to the functionality and the arrangement of the components described, can be made without deviating from the scope of protection as it results from the claims and combinations of features equivalent thereto.

### LIST OF REFERENCE SIGNS

1 body irradiation device
2 irradiation module
3, 3a, 3b circuit board
4 first LED source of radiation (UV-A radiation)
5 second LED source of radiation (UV-B radiation)
6a, 6b electric circuits for first LED sources of radiation in series connection
7 electric circuit for second LED sources of radiation in series connection
8, 9 contacts 8, 9 of the electric circuits 6a, 6b
11 third LED source of radiation (red light or IR light)
12 electric circuit according to FIG. 3
15 panel
16 plastic panel
17 exposure tunnel
18 upper part of the body irradiation device
19 lower part of the body irradiation device 1
20 ring arrangement which covers the first LED sources of radiation
22 heat sink
23 bridge in order to lead the further electric circuit 7 across the electric circuits 6a, 6b
24 ring arrangement which covers the second LED sources of radiation.
25 control means
26a, 26b user interface
27a, 27b, 27c, 27d, 27e scenario of irradiation
28a, 28b, 28c irradiation profile
29 fourth LED sources of radiation
30 first regions
31 second regions
32a, 32b section
33 housing
34a, 34b end face
35 transparent surface
36 joint
37a, 37b frame
38a, 38b fan for added comfort
39 base
40 pivot arm
41a, 41b support arm
42 cooling fins
43 power supply
44 slide controller

1. A body irradiation device for applying actinic radiation to a living organism, in particular a human being, wherein the body irradiation device comprises:
at least one irradiation module, wherein the at least one irradiation module comprises:
first LED sources of radiation which are adapted to emit UV-A radiation, and second LED sources of radiation which are adapted to emit UV-B radiation; and
a control means for controlling the first LED sources of radiation and the second LED sources of radiation via their respective electric circuits in such a way that a specific radiation intensity and/or a specific radiation dose of UV-A radiation and a specific radiation intensity and/or a specific radiation dose of UV-B radiation are emitted.

2. The body irradiation device according to claim 1, wherein the at least one irradiation module further comprises:
at least one first circuit board on which the first LED sources of radiation are arranged; and
at least one second circuit board on which the second LED sources of radiation are arranged;
wherein the first circuit board has first regions and the second circuit board has second regions, wherein the first regions overlap with the second regions, and
wherein a first LED source of radiation is arranged in at least one first region and a second LED source of radiation is arranged in at least one second region.

3. The body irradiation device according to claim 2, wherein the at least one first circuit board and the at least one second circuit board are arranged substantially in the longitudinal direction of the body irradiation device in an alternating manner,
wherein the first circuit board has at least one first electric circuit and wherein the second circuit board has at least one second electric circuit, wherein the at least one first electric circuit connects the first LED sources of radiation among one another, wherein the at least one second electric circuit connects the second LED sources of radiation among one another, and wherein the circuit boards have separate electrical connections for the at least one first electric circuit and the at least one second electric circuit.

4. (canceled)

5. (canceled)

6. The body irradiation device according to claim 1, wherein the control means is arranged to control the first LED sources of radiation and the second LED sources of radiation in different sections in the longitudinal direction of the body irradiation device, in particular on different circuit boards, in such a way that different radiation intensities and/or radiation doses are emitted,
wherein the body irradiation device further comprising an exposure tunnel in which a user can lie down in order to be irradiated with actinic radiation,
wherein the exposure tunnel is closed by substantially pivoting an upper part of the body irradiation device towards a lower part of the body irradiation device,
wherein the lower part of the body irradiation device has an at least substantially transparent surface, under which irradiation modules are arranged; and
wherein irradiation modules are also arranged on the upper part.

7. (canceled)

8. The body irradiation device according to claim 1, which has a greater number of first LED sources of radiation than second LED sources of radiation,
wherein the number of first LED sources of radiation is selected in such a way that an operating voltage of the at least one first electric circuit does not exceed about 70 V, and/or wherein and the number of second LED sources of radiation is selected in such a way that an operating voltage of the at least one second electric circuit does not exceed about 60 V.

9. (canceled)

10. The body irradiation device according to claim 1, wherein the at least one irradiation module further comprises:
a transparent panel which extends over the first LED sources of radiation and the second LED sources of radiation, wherein the panel is spaced apart from the circuit board, and at least a side of the panel which faces away from the circuit board is sanitized,
wherein the transparent panel is the only optical element of the at least one irradiation module, wherein the transparent panel is a glass panel or an acrylic panel.

11. (canceled)

12. (canceled)

13. The body irradiation device according to claim 1, wherein a radiation angle of the first LED sources of radiation and/or of the second LED sources of radiation does not exceed about 50 degrees,
wherein the first LED sources of radiation and the second LED sources of radiation are controlled in such a way that the radiation intensity of the first LED sources of radiation and/or a radiation intensity of the second LED sources of radiation varies over time.

14. (canceled)

15. The body irradiation device according to claim 1, further comprising a sensor which is set up to measure at least one physiological parameter, in particular a pigmentation and/or a reaction of the skin of the living organism to an irradiation dose, wherein the first LED sources of radiation and the second LED sources of radiation are controlled in such a way that the radiation intensity or radiation intensities varies or vary as a function of the at least one physiological parameter.

16. The body irradiation device according to claim 1, further comprising a user interface which is set up in such a way that a radiation intensity of the UV-A radiation to be emitted and/or a radiation dose of the UV-A radiation to be emitted and a radiation intensity of the UV-B radiation to be emitted and/or a radiation dose of the UV-B radiation to be emitted can be set by means of the user interface individually as a function of a maximum permissible erythema effective radiation intensity of UV radiation, and
wherein the first LED sources of radiation and the second LED sources of radiation are controlled on the basis of a selection, at the user interface, of the radiation intensity of the UV-A radiation to be emitted and/or the radiation dose of the UV-A radiation to be emitted and the radiation intensity of the UV-B radiation to be emitted and/or the radiation dose of the UV-B radiation to be emitted,
wherein a temporal course of the radiation intensity of the UV-A radiation to be emitted and a temporal course of the radiation intensity of the UV-B radiation to be emitted can additionally be set via the user interface, and wherein the first LED sources of radiation and the second LED sources of radiation are additionally controlled on the basis of a selection of a temporal course.

17. (canceled)

18. The body irradiation device according to claim 1, further comprising a user interface,
wherein at least one scenario of irradiation is stored in the means for controlling, for which a maximum permissible erythema effective radiation intensity of UV radiation is defined and which comprises a plurality of irradiation profiles,
wherein the plurality of irradiation profiles each define a radiation intensity of the UV-A radiation to be emitted and/or a radiation dose of the UV-A radiation to be emitted and a radiation intensity of the UV-B radiation to be emitted and/or the radiation dose of the UV-B radiation to be emitted as a function of the maximum erythema effective radiation intensity of UV radiation,
wherein the user interface is configured such that the plurality of irradiation profiles are arranged to be selected, and wherein the first LED sources of radiation and the second LED sources of radiation are controlled on the basis of a selection, at the user interface, of one of the plurality of radiation profiles,
wherein a plurality of scenarios of irradiation are stored in the means for controlling, which scenarios of irradiation can be selected by means of the user interface, wherein, for each scenario of irradiation, a different maximum permissible erythema effective radiation intensity of UV radiation is defined,
wherein the irradiation profiles additionally define, via the user interface, a temporal course of the radiation intensity of the UV-A radiation to be emitted and a temporal course of the radiation intensity of the UV-B radiation to be emitted.

19. (canceled)

20. (canceled)

21. The body irradiation device according to claim 18, wherein the user interface is configured such that the plurality of irradiation profiles are arranged to be selected, in particular continuously, between a maximum radiation profile with the highest radiation intensity to be emitted and/or with the highest radiation dose to be emitted and at least one radiation profile with a lower radiation intensity to be emitted and/or with the lower radiation dose to be emitted,
wherein the radiation intensity of the UV-A radiation to be emitted and/or the radiation dose of the UV-A radiation to be emitted varies between different irradiation profiles differently than the radiation intensity of the UV-B radiation to be emitted and/or the radiation dose of the UV-B radiation to be emitted.

22. (canceled)

23. The body irradiation device according to claim 18, wherein the body irradiation device has at least two of the irradiation profiles from the following group of irradiation profiles: In The Morning—Intensive, In The Morning—Medium, In The Morning—Sensitive, At Midday—Intensive, At Midday—Medium, At Midday—Sensitive, In The Evening—Intensive, In The Evening—Medium and In The Evening—Sensitive, wherein, at least substantially, the maximum permissible erythema effective radiation intensity of UV radiation is reached at 100% UV-A radiation and 100% UV-B radiation, wherein the irradiation profiles are defined in the following table:

| | | In The | | In The |
|---|---|---|---|---|
| | | Morning | At Midday | Evening |
| Intensive | UV-A | about 75-85% | about 95-100% | about 75-85% |
| | UV-B | about 55-65% | about 95-100% | about 55-65% |
| Medium | UV-A | about 65-75% | about 85-95% | about 65-75% |
| | UV-B | about 45-55% | about 85-95% | about 45-55% |
| Sensitive | UV-A | about 55-65% | about 85-95% | about 55-65% |
| | UV-B | about 35-45% | about 75-85% | about 35-45% |

24. (canceled)

25. The body irradiation device according to claim wherein the at least one irradiation module further comprises:
third LED sources of radiation, which are constructed so as to emit red radiation and/or infrared radiation,
wherein the control means is arranged to control the third LED sources of radiation in such a way that a specific radiation intensity and/or a specific radiation dose of red radiation and/or infrared radiation is emitted,
wherein the irradiation profiles from the group of irradiation profiles are further defined as follows, wherein, at least substantially, the maximum permissible radiation intensity of red radiation and/or infrared radiation is reached at 100% red radiation:

| | | In The | | In The |
|---|---|---|---|---|
| | | Morning | At Midday | Evening |
| Intensive | Red | about 25-35% | about 95-100% | about 95-100% |
| Medium | Red | about 25-35% | about 95-100% | about 95-100% |
| Sensitive | Red | about 25-35% | about 95-100% | about 95-100% |

26. The body irradiation device according to claim 1, wherein the at least one irradiation module further comprises: fourth LED sources of radiation, which are constructed so as to emit radiation in the visible spectrum and which are connected in series with the second LED sources of radiation in a second electric circuit and which have the same power supply as the second LED sources of radiation, and
wherein the fourth LED sources of radiation are configured to emit radiation in the visible spectrum, and which are controlled together with the second LED sources of radiation in such a way that the fourth LED sources of radiation are activated when the second LED sources of radiation are activated.

27. (canceled)

28. (canceled)

29. The body irradiation device according to claim 1, wherein, in a section of the irradiation module in the longitudinal direction of the body irradiation device in which a face of the living organism is arranged during use as intended, more first LED sources of radiation and second LED sources of radiation are arranged than in other sections of the body irradiation device,
wherein the number of second LED sources of radiation of the at least one irradiation module is selected in such a way that they can be operated at less than 70 percent of the rated current or rated power of the second LED sources of radiation in order to emit the specific radiation intensity of UV-B radiation and/or to emit the specific radiation dose of UV-B radiation in a predetermined period of time.

30. (canceled)

31. The body irradiation device according to claim 1, wherein the at least one irradiation module has a housing with end faces, wherein, on the end faces, regions which are permeable to air are provided for air supply, and, in a central area of the housing, an opening is provided for the discharge of air,
wherein the opening of the housing of the at least one irradiation module is connected to an air duct of the body irradiation device, and wherein the air duct leads to a fan in the lower part of the body irradiation device.

32. (canceled)

33. A non-therapeutic method for the application of actinic radiation to a living organism, in particular a human being, by means of a body irradiation device, in particular according to any one of the preceding claims, wherein the method comprises the following process step:
controlling at least one first electric circuit and of the at least one second electric circuit in such a way that a specific radiation intensity and/or a specific radiation dose of UV-A radiation and a specific radiation intensity and/or a specific radiation dose of UV-B radiation are emitted,
wherein the at least one first electric circuit connects first LED sources of radiation which are configured to emit UV-A radiation, among one another, and
wherein the at least one second electric circuit connects second LED sources of radiation, which are configured to emit UV-B radiation, among one another.

34. The method according to claim 33, wherein the electric circuits are controlled in such a way that the radiation intensity of the first LED sources of radiation of the at least one first electric circuit and/or a radiation intensity of the second LED sources of radiation of the at least second electric circuit varies over time.

35. The method according to claim 33, further comprising the following process steps:
measuring at least one physiological parameter, in particular a pigmentation and/or a reaction of the skin to an irradiation dose, of the living organism, wherein the radiation intensity or the radiation intensities varies or vary as a function of the at least one physiological parameter;
detecting a selection, in particular an individual selection, of a radiation intensity of the UV-A radiation to be emitted and/or a radiation dose of the UV-A radiation to be emitted and a radiation intensity of the UV-B radiation to be emitted and/or a radiation dose of the UV-B radiation to be emitted, as a function of a maximum permissible erythema effective radiation intensity of UV radiation:
wherein the first LED sources of radiation and the second LED sources of radiation are controlled on the basis of the selection of the radiation intensity of the UV-A radiation to be emitted and/or the radiation dose of the UV-A radiation to be emitted and the radiation intensity of the UV-B radiation to be emitted and/or the radiation dose of the UV-B radiation to be emitted;
providing at least one scenario of irradiation for which a maximum permissible erythema effective radiation intensity of UV radiation is defined and which comprises a plurality of irradiation profiles;
detecting a selection of an irradiation profile from the plurality of irradiation profiles, each of which define a radiation intensity of the UV-A radiation to be emitted and/or a radiation dose of the UV-A radiation to be emitted and a radiation intensity of the UV-B radiation to be emitted and/or the radiation dose of the UV-B radiation to be emitted, as a function of the maximum permissible erythema effective radiation intensity of UV radiation;
wherein the first LED sources of radiation and the second LED sources of radiation are controlled on the basis of the selection of the one of the plurality of irradiation profiles; and
detecting a selection of a scenario of irradiation from a plurality of scenarios of irradiation, wherein, for each scenario of irradiation, a different maximum permissible erythema effective radiation intensity of UV radiation is defined.

36. (canceled)

37. (canceled)

38. (canceled)

39. The method according to claim 33, wherein the second LED sources of radiation are operated at less than 70 percent of the rated current or the rated power of the second LED sources of radiation,
wherein the first LED sources of radiation and the second LED sources of radiation in different sections in the longitudinal direction of the body irradiation device, on different circuit boards, are controlled in such a way that different radiation intensities and/or radiation doses are emitted in different sections.

40. (canceled)
